(19) United States
(12) Patent Application Publication
(10) Pub. No.: US 2015/0011826 A1
(43) Pub. Date: Jan. 8, 2015

(54) MEDICO-SURGICAL ASSEMBLIES

(71) Applicant: SMITHS MEDICAL INTERNATIONAL LIMITED, Kent (GB)

(72) Inventors: Christopher Paul Mayo, Horsham (GB); Mark Charles Oliver, St. Albans (GB); Eric Pagan, Hythe (GB)

(73) Assignee: SMITHS MEDICAL INTERNATIONAL LIMITED, Kent (GB)

(21) Appl. No.: 14/379,352

(22) PCT Filed: Jan. 24, 2013
(86) PCT No.: PCT/GB2013/000025

§ 371 Date: Aug. 18, 2014

(30) Foreign Application Priority Data
Feb. 25, 2012 (GB) 1203389.0

(51) Int. Cl. A61M 16/04; A61B 1/267; A61B 1/00

(52) U.S. Cl. CPC A61M 16/0488; A61B 1/00124; A61B 1/0011; A61M 16/0434; A61B 1/267; A61M 2205/502
USPC 600/114

(57) ABSTRACT

A video bougie 1 for use in inserting a medical tube 3 includes a bendable rod 10 with a camera unit 11 mounted at its forward end and connected by a cable 13 with an electrical connector 14 at the machine end of the bougie. A brass ferrule 30 has its forward end 32 attached to an external recess 24 around the machine end of the rod 10, the rear end 33 of the ferrule supporting the electrical connector 14.

This invention relates to medico-surgical assemblies of the kind including a bendable elongate member, an imaging sensor at a patient end of the assembly, an electrical connector at a machine end of the assembly, and an electrical cable extending within the member along its length connected with the imaging sensor at its patient end and with the electrical connector at the machine end of the assembly.

The invention is more particularly concerned with medico-surgical assemblies for use in placing apparatus within a patient such as inserting an endotracheal tube into the trachea. Traditional introducers or bougies take the form of a simple rod that can be bent to an approximate desired shape and can flex to accommodate the shape of the anatomy during insertion. The introducer may be made with an angled, Coude tip to facilitate introduction. The introducer can be inserted more easily than the tube itself because it has a smaller diameter and can be bent and flex to the ideal shape for insertion. The small diameter also gives the clinician a better view of the trachea around the outside of the introducer. These may be used with or without the aid of a laryngoscope. When the bougie has been correctly inserted, a tube can be slid along the outside of the bougie to the correct location, after which the bougie is pulled out of the tube, which is left in position. Bougies are available from Smiths Medical. GB2312378 describes an introducer or bougie moulded of an aliphatic polyurethane material and also describes an earlier bougie made from a braided polyester filament repeatedly coated in layers of resin. The characteristic flexural and recovery properties of these bougies are highly valued by clinicians.

More recently it has been proposed to use fibre optics or a CCD camera with an introducer to provide the clinician with a view of the trachea as the introducer is inserted. WO2007/089491 describes an arrangement with an introducer and a separate camera assembly clipped onto the outside of the introducer, which is removed before an endotracheal tube can be slid along the introducer. Alternative arrangements have a handle at the machine end of the introducer so that the apparatus has to be inserted with the tube already loaded on the introducer from its patient end. Such arrangements are more difficult to use because of the presence of the tube during insertion. WO2010/136748 describes an introducer arrangement with a camera at one end of an introducer and connected via a cable to a display screen at the opposite end. The introducer is disconnected from the display when correctly positioned so that an endotracheal tube can be slid along the introducer into position, following which the introducer is removed by pulling rearwardly from the endotracheal tube.

It is particularly important that these video introducers or bougies retain the flexural and recovery properties of conventional bougies, even after sterilisation and prolonged storage. If the diameter of the bore along the bougie has to be enlarged this can adversely affect the handling properties of the bougie. Another problem is that it can be difficult to connect the cable at the rear end of the bougie with an electrical connector. If the cable length is extended beyond the bougie to allow access for connection to a connector there can be surplus cable but no space within the bougie to receive this.

It is an object of the present invention to provide alternative medico-surgical assemblies.

According to one aspect of the present invention there is provided a medico-surgical assembly of the above-specified kind, characterised in that the assembly includes a tubular extension piece attached with the outside of the member at its machine end, the extension piece providing an enlarged internal cavity to accommodate a part of the length of the cable, and that the electrical connector is supported on the extension piece.

The elongate member may be of a plastics material and the extension piece of a metal. The forward end of the extension piece is preferably secured on a recess at the rear end of the elongate member. The cable may include a screening sleeve along its length within the elongate member up to the forward end of the extension piece, the extension piece providing screening of wires in the cable along that length within the extension piece. The assembly may include processing means within the extension piece connected between the cable and the connector.

According to another aspect of the present invention there is provided a bougie for use in introducing a medical tube into a patient, the bougie including a bendable rod of a plastics material supporting an imaging sensor at the patient end of the bougie, an electrical cable extending along a passage through the rod from the sensor and an electrical connector at the machine end of the bougie, characterised in that the machine end of the rod has a portion with a reduced external diameter, an extension piece fitted on the portion of reduced diameter and extending beyond the machine end of the rod, the extension piece having an internal diameter greater than that of the passage along the rod, and that the electrical connector is connected with a machine end of the cable and is supported by a machine end of the extension piece.

According to a further aspect of the present invention there is provided a method of making a video introducer including the steps of providing a bendable elongate member having a passage extending along its length, providing an image sensor attached with a forward end of an electrical cable, threading a rear end of the cable along the passage so that it protrudes from the machine end of the elongate member, connecting the machine end of the cable with an electrical connector, applying an extension piece around the machine end of the cable with the forward end of the extension piece extending along a region of the outside of the elongate member at its machine end, and attaching the connector with the rear end of the extension piece.

The extension piece may be slid forwardly over the connector onto the region at the machine end of the elongate member after connecting the machine end of the cable with the electrical connector. Alternatively, the extension piece may be slid forwardly along the machine end of the cable onto the region at the machine end of the elongate member, and that the machine end of the cable is subsequently attached to the electrical connector.

According to a fourth aspect of the present invention there is provided a video introducer made by a method according to the above further aspect of the present invention.

An endotracheal tube placement assembly according to the present invention will now be described, by way of example, with reference to the accompanying drawings, in which:

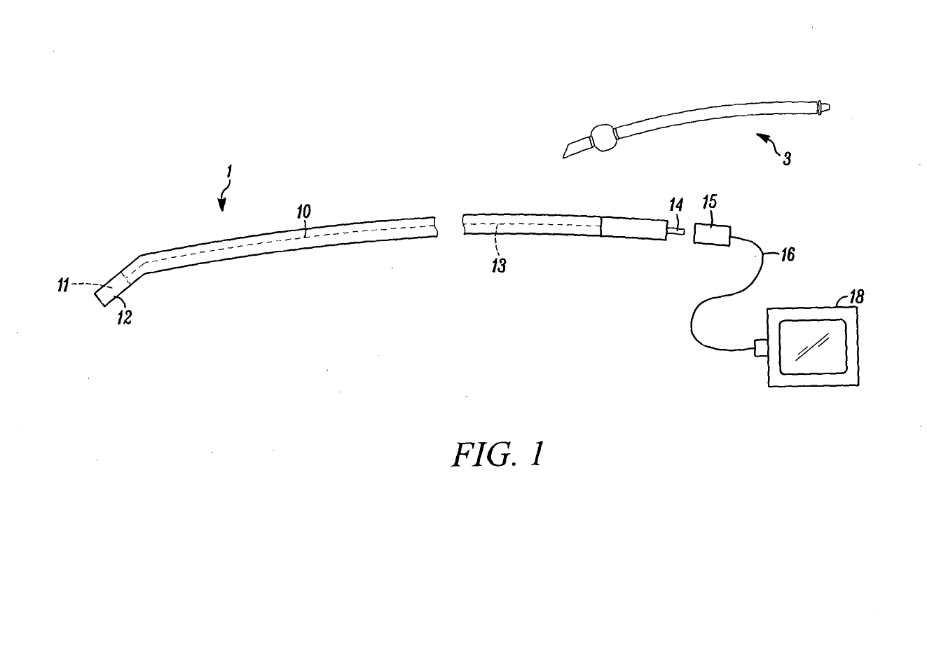

FIG. 1 shows an assembly of viewing means in the form of a video introducer and an endotracheal tube;

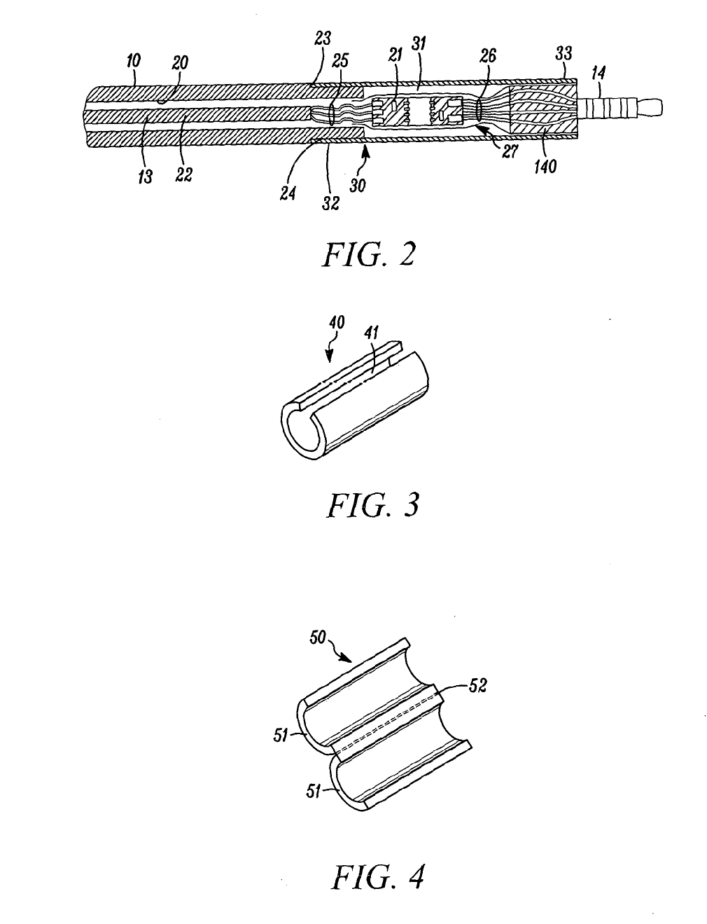

FIG. 2 is an enlarged cross-section view of the rear or machine end of the assembly of FIG. 1;

FIG. 3 is a perspective view showing a first alternative extension piece for the assembly; and

FIG. 4 is a perspective view showing a second alternative extension piece for the assembly.

With reference first to FIGS. 1 and 2, the assembly consists of viewing means or a video introducer 1 in the form of a flexible, bendable plastics elongate member, rod or bougie 10 with a small camera and illumination unit 11 mounted at its forward patient end 12. The camera and illumination unit 11 are connected with one end of an electrical cable 13 the opposite end of which is connected with a low profile electrical connector 14, which is small enough to allow an endotracheal tube 3 to be slid over the connector. The connector 14 mates removably with a connector 15 attached at one end of a second cable 16 extending to a video display monitor 18 on which is shown a display representation of the illuminated field of view of the camera unit 11.

The camera unit 11 is pre-connected at the forward or patient end of the cable 13 so that the rear or machine end of the cable can be threaded along a bore or passage 20 of the rod 10, the length of the cable being greater than that of the rod so that sufficient length of cable projects from the machine end of the rod to enable it to be connected directly to the connector 14, or as shown in FIG. 2, to be connected to the connector via an image processing unit on a small pcb 21. The cable 13 is electrically screened along most of its length by a braided sleeve or similar jacket 22. The braided sleeve 22 terminates a short distance before the end of the rod 10, level with a shallow step 23 at one end of a reduced diameter recess 24 machined around the outside of the rod. For a rod 10 with an external diameter of 5 mm the recess 24 is typically between 5 mm and 10 mm long. The individual wires 25 of the cable 13 extend freely beyond the screen 22 to enable them to be soldered or otherwise electrically connected to contacts at the forward end of the pcb 21. Contacts at the rear end of the pcb 21 are connected to the forward end 140 of the connector 14 by wires 26. A heat shrink sleeve 27, such as sold by 3M under the code GTI 3000 or by Cleevetech under the code M053-5C1, is shrunk about the pcb 21, the forward end of the connector 14 and a part of the length of the wires 25.

The introducer 1 is completed by an extension piece in the form of a cylindrical brass ferrule 30 about 40 mm long and having an external diameter the same as that of the bougie rod 10. The ferrule 30 has an internal cavity 31 defined by a constant internal diameter along its length equal to the external diameter of the recess 24 at the rear end of the bougie rod 10. The diameter of the cavity 31 is, therefore, appreciably greater than the diameter of the passage 20 through the rod 10. The forward end 32 of the ferrule 30 extends along the recess 24 to abut the step 23 and is secured on the rod 10 such as by an adhesive. The electrical connector 14 is supported at the opposite, rear end 33 of the ferrule 30. The external diameter of the forward part 140 of the connector is a close sliding fit within the rear end of the ferrule 30 so that it can be secured with the ferrule by an adhesive. The dimensions of the ferrule 30 are such that the cable 13 can be connected with the pcb 21, and the pcb connected with the connector 14 before assembly of the ferrule, which can be slid into place on the rear end of the rod 10 along the outside of the connector. Alternatively, the ferrule 30 could be assembled before the connector 14 with sufficient length of wires 25 and 26 being provided to allow these to extend beyond the rear end 33 of the ferrule for connection. The volume of the cavity 31 within the ferrule 30 allows room to accommodate the excess length of the wires when the connector 14 is pushed into the ferrule for attachment.

The provision of the separate extension piece greatly facilitates electrical connection at the rear end of the bougie. Where the ferrule is made of brass or other metal it provides electrical screening of the pcb and of the wires extending beyond the screening along the cable.

It is not essential for the extension piece to be in the form of a metal ferrule since it could be of other materials and forms. FIG. 3 shows an extension piece 40 of an extruded of moulded plastics material having a tubular shape with a longitudinal slit 41 along its length so that it can be enlarged slightly diametrically to allow the pcb 21 and the wires 25 and 26 to be slid in laterally and for it to be extended over the recess 24 on the rod 10 and the connector 14. The slit 41 is subsequently closed by an adhesive, filler or the like.

FIG. 4 shows a slightly different extension piece 50 in the form of a single-piece, unitary plastics casting having two hemi-cylindrical shell members 51 linked along their length on one side by a living hinge 52. This piece can be folded about the rear end 24 of the rod 10 and the connector 14 to enclose the wires 25 and 26 and the pcb 21. An adhesive or other bond is used to form a permanent attachment along the free sides of the two shell members and between the extension piece and the rod 10 and connector 14. Alternatively, or in addition to the adhesive, the hinged extension piece could have a snap fit mechanism on the two shell members so that, when folded together they mechanically lock.

The two extension pieces 40 and 50 could be made electrically conductive for screening purposes such as by loading the material of the pieces with a conductive filler or by coating with a conductive layer, by using a conductive adhesive, paint or a metal foil lining.

In another arrangement, the extension piece could be provided by casting a plastics or resin material in situ about the rear end of the cable 13, the pcb and the forward end of the connector.

1-10. (canceled)

11. A medico-surgical assembly including a bendable elongate member, an imaging sensor at a patient end of the assembly, an electrical connector at a machine end of the assembly, and an electrical cable extending within the member along its length connected with the imaging sensor at its patient end and with the electrical connector at the machine end of the assembly, characterized in that the assembly includes a tubular extension piece attached with the outside of the member at its machine end, the extension piece providing an enlarged internal cavity to accommodate a part of the length of the cable, and that the electrical connector is supported on the extension piece.

12. An assembly according to claim 11, characterized in that the elongate member is of a plastics material and the extension piece is of a metal.

13. An assembly according to claim 11, characterized in that the forward end of the extension piece is secured on a recess at the rear end of the elongate member.

14. An assembly according to claim 11, characterized in that the cable includes a screening sleeve along its length within the elongate member up to the forward end of the extension piece, and that the extension piece provides screening of wires in the cable along that length within the extension piece.

15. An assembly according to claim 11, characterized in that the assembly includes processing means within the extension piece connected between the cable and the connector.

16. A bougie for use in introducing a medical tube into a patient, the bougie including a bendable rod of plastics material supporting an imaging sensor at the patient end of the bougie, an electrical cable extending along a passage through the rod from the sensor and an electrical connector at the machine end of the bougie, characterized in that the machine end of the rod has a portion with a reduced external diameter, an extension piece fitted on the portion of reduced diameter and extending beyond the machine end of the rod, the extension having an internal diameter grater than that of the passage along the rod, and that the electrical connector is connected with a machine end of the cable and is supported by a machine end of the extension piece.

17. A method of making a video introducer including the steps of providing a bendable elongate member having a passage extending along its length, providing an image sensor attached with a forward end of an electrical cable, threading a rear end of the cable along the passage so that it protrudes from the machine end of the elongate member, connecting the machine end of the cable with an electrical connector, applying an extension piece around the machine end of the cable with the forward end of the extension piece extending along a region of the outside of the elongate member at its machine end, and attaching the connector with the rear end of the extension piece.

18. A method according to claim 17, characterized in that the extension piece is slid forwardly over the connector onto the region at the machine end of the elongate member after connecting the machine end of the cable with the electrical connector.

19. A method according to claim 17, characterized in that the extension piece is slid forwardly along the machine end of the cable onto the region at the machine end of the elongate member, and that the machine end of the cable is subsequently attached to the electrical connector.

20. A video introducer made by a method according to claim 17.

21. An assembly according to claim 12, characterized in that the forward end of the extension piece is secured on a recess at the rear end of the elongate member.
